(19) United States
(12) Patent Application Publication
(10) Pub. No.: US 2026/0114748 A1
(43) Pub. Date: Apr. 30, 2026

(54) Two Speed System for Collecting and Measuring a Dynamic Signal

(71) Applicant: Anthony David Wondka, San Ramon, CA (US)

(72) Inventor: Anthony David Wondka, San Ramon, CA (US)

(73) Assignee: Anthony Wondka, San Ramon, CA (US)

(21) Appl. No.: 18/931,021

(22) Filed: Oct. 29, 2024

(51) Int. Cl. A61B 5/08; A61B 10/00; G01N 33/497

(52) U.S. Cl. CPC A61B 5/082; A61B 10/00; G01N 33/497; A61B 2010/0087

(57) ABSTRACT

A system and method are described for measuring a characteristic in a dynamic signal, such as a physiologic signal, that has a frequency that is too fast to permit accurate measurement of the characteristic. The specimen containing or producing the signal is collected at a relatively high speed that is selected to yield an ample quantity of sample for measurement. The specimen is then measured at a slower speed which is selected based on the response time of the measurement sensor, such that the signal frequency is slowed down enough during measurement to be near to or slower than the sensor's response time, thus allowing for a precise and accurate measurement of the characteristic.

### CROSS-REFERENCE TO RELATED ABANDONED APPLICATIONS

For disclosure purposes, this non-provisional patent application is a new filing of abandoned provisional patent applications 63/546,228 filed Oct. 29, 2023, and 63/389,933 filed Jul. 17, 2022; and abandoned U.S. patent application Ser. No. 17/476,439 filed Sep. 15, 2021 which was never published, which claimed priory to provisional patent application 63/078,819 filed Sep. 15, 2020. A petition for express abandonment without publication for application Ser. No. 17/476,439 was submitted by the Applicant on May 21, 2022 and granted by the USPTO. The title, specification, figures, and inventorship in this new non-provisional patent filing are unchanged from 63/546,228, 63/389,933, Ser. No. 17/476,439, and 63/078,819, and these aforementioned abandoned applications have never been publicly disclosed as of the time of this new filing. The priority date of this new Application should therefore be the date of this filing.

### BACKGROUND

The accurate measurement of some dynamic systems can be technically and scientifically challenging because a frequency in the system is faster than the technology that is available to measure a characteristic signal of the system that is desired to be measured. This challenge can exist in many physical systems in natural life, or in synthetic systems. One area for example is that of physiology, such as but not limited to electrophysiology signals, neurological signals, cardiac signals, vascular signals, cellular and biochemical processes, respiratory signals and breath signals. Specimens may be biological samples, images, electrical recordings, sound, movement, or other specimens. In the following, we shall use the example of breath samples and signals in the breath to describe this invention, in particular measuring the composition of a gas in an exhaled breath sample, however, this should be construed as exemplary only for purposes of brevity, and it should be understood that the invention applies to the measurement of high frequency signals in all other dynamic systems.

Neonates breathe at 45 breaths per minute on average when at rest, but when neonates are stressed, premature, or ill, they may indeed breathe as high as 70 breaths per minute on a sustained basis. Each breath cycle consisting of one inhalation and one exhalation can therefore have a 0.86 second period, with the exhalation portion being around 0.43 seconds. One important vital sign to measure in some neonates is the exhaled CO2, which provides an important glimpse into how well the patient is breathing, exchanging gas, respirating, ventilating and perfusing, as well as levels of sedation during anesthesia. It is an important physiological parameter to measure in order to know the status of various clinical issues. CO2 from the alveoli, the gas exchange compartment of the lung where the gas composition can be equated to that of the blood stream on the other side of the lung-blood barrier, begins to exit the nose after about half of the tidal volume is exhaled. This occurs about half way through the exhalation time, at which time the CO2 concentration exiting the nose will rise from roughly zero to that of the alveolar concentration, its peak level, in about 0.100 seconds. While the underlying physics of the CO2 sensor respond relatively quick, in about 0.050 seconds, the overall system of the sensor adds to that base response time, making the overall response time much greater. The best available sensor technology for measuring the CO2 in the breath in real time has an overall response time of about 0.150 to 0.300 seconds. Therefore, at the moment that the peak CO2 level occurs, the sensor is considerably behind the actual CO2 level, and registers only a fraction of the peak CO2 level. And very soon after peak CO2 level is reached, the neonate begins the next inspiration, no longer exhaling CO2, and therefore not giving the CO2 sensor the extra time it requires to catch up to that peak CO2 level that occurred moments ago. A similar fast breath rate challenge exists in veterinary medicine, for example with rabbits and other species, whether it be in the clinical care or in the research setting. Similar challenges exist in other physiologic and non-physiologic systems.

Many manufacturers of CO2 monitors (or capnometers) have not attempted to addressed this issue, and are resigned to the fact that their monitor is only good for measuring breath rate (a secondary function of a capnometer) under these conditions and are not good for accurate CO2 levels. However, some equipment manufacturers have attempted to address this issue and have created extrapolation algorithms that, based on the sensor's measurement signal slope, albeit temporally well behind the true CO2 level at any instant in time, extrapolate where the CO2 signal would have peaked, and report that extrapolated value instead of the sensor's true measurement. This approach should not be used, because the potentially complex clinical issues related to a neonate's breathing will undoubtedly eventually fool the algorithm, giving the user untrustworthy data.

The options to overcome this challenge, rather than measuring exhaled CO2, are either (a) performing paCO2 measurements on blood samples, or (b) performing transcutaneous CO2 measurements through the skin. Blood gas measurements cannot be performed frequently or fast enough to meet the clinical need, even with point of care systems which provide the result in a few minutes, because it is too traumatic to obtain the specimen. There have been attempts to develop in-dwelling continuous blood gas monitoring systems, however these systems have technical and usability challenges, and after four decades of efforts, are still not mainstream in neonatal care. Transcutaneous monitoring of CO2 is showing some adoption promise, however, as more of a trending tool, not a diagnostic measurement, because of the reported inaccuracies and because some situations do not lend themselves to placement of the transcutaneous sensor on the patient. If ever possible, measuring exhaled CO2 would be the preferred modality in many clinical scenarios for monitoring CO2 levels. It can be concluded therefore that there is unquestionably an unmet need in the field of neonatal CO2 monitoring, and there may also be unmet needs in the broader field of measuring dynamically fast signals.

In this present invention, a solution is described which solves the technical and scientific challenges of accurately and precisely measuring CO2 in the exhaled breath of fast breathing patients, especially neonates. Specifically, an effectively-continuous, real-time CO2 monitor has been invented which is capable of accurately and precisely measuring the CO2 level in the exhaled breath, at virtually any breath rate, using a true, direct measurement without the need for any extrapolation, correction or compensation. In a first main embodiment of the invention, an apparatus collects a breath sample at a relatively fast collection speed, and immediately after collection without pause, plays the collected sample through a CO2 sensor at a slower speed that is suitable for the sensor's response time, allowing the sensor to keep up with the actual CO2 level in the sample as the sample is traveling through the sensor. The invention as it applies to neonatal end-tidal CO2 monitoring, may be referred to as the “Neo-CAP™” device with “Dual Track Technology™”. While CO2 measurements are described for one implementation of this invention, it should be understood that the invention explicitly and implicitly applies to measuring other gases in the exhaled gas, as well as other dynamically fast physiological and non-physiological signals. Detailed aspects of this main embodiment, and secondary embodiments of the invention will become apparent in the following descriptions.

Relevant prior art for this invention could not be found. The closest prior art which is, albeit not relevant, can be found in the fields of video and sound recording, and in the fields of power and communications transmissions. For example, it is common practice to record a video or audio piece, and after recorded, play the piece back at a slower speed for editing or viewing purposes. With regard to dynamic signal measurements however, especially physiological dynamic signal measurements including breath measurements, no relevant prior art could be found. In some breath analysis prior art, a specimen is collected and stored for a while, and eventually measured for a one-time measurement. There is no mention of measuring at a different speed than that which it was collected, nor are these systems capable of continuous monitoring—they perform one-point measurements. In capnometry prior art, real time continuous measuring systems are described that collect and measure the breath sample at a certain speed, but there is no purpose stated behind the speed selected which is somewhat arbitrary and there is certainly no mention of measuring at a slower speed than the collected speed.

### BRIEF DESCRIPTION OF THE DRAWINGS

FIG. 1 is a pneumatic schematic diagram of a main embodiment of the invention showing two parallel flow paths, each with two branches.

FIG. 2 is a timing diagram of the system in FIGS. 1, 3 and 4, showing the pump status, valve control, flow path routing, and sensor output as a function of time.

FIG. 3 is a pneumatic schematic of the system in FIG. 1 showing path 1 in collection mode and simultaneously path 2 in measurement mode.

FIG. 4 is a pneumatic schematic of the system of FIG. 1 with path 2 in collection mode and path 1 in measurement mode.

FIG. 5 is a graph of the signal level versus time, comparing actual signal to conventional sensor output and the present invention output.

FIG. 6 shows time-based plots of a conventional sensor output and the invention's sensor output in comparison to actual values from a series of breaths from a patient.

FIG. 7 is a pneumatic schematic diagram of an alternative system having a valve-less input section upstream of the sensor, shown with state 1 active.

FIG. 8 is a pneumatic schematic diagram of an alternate system using one-channel zero dead space pincher valves in the input section upstream of two parallel sensors.

FIG. 9 is a pneumatic schematic diagram of an alternate system using one-channel zero dead space pincher valves in place of the 3-way valves of the system shown in FIG. 1.

FIG. 10 is a pneumatic schematic diagram of an alternate system using two-channel zero dead space pincher valves replacing the 3-way valves of the system in FIG. 1, and a single measurement sensor, shown in flow state 1.

FIG. 11 is a pneumatic schematic diagram of the system shown in FIG. 10, shown in flow state 2.

FIG. 12a is a front view line drawing of the two-channel pincher valve used in the system of FIGS. 10 and 11, shown in the neutral both channels open condition.

FIG. 12b is a front view line drawing of the valve of FIG. 12a shown in the channel A open, channel B closed position.

FIG. 12c is a front view line drawing of the valve of FIG. 12a shown in the channel A closed, channel B open position.

FIG. 13 is a pneumatic schematic diagram of an alternate system using parallel sensors and showing exemplary flow path resistance values.

FIG. 14 is a pneumatic schematic diagram of an alternate system using a combination of 3-way valves and zero dead space pincher valves.

FIG. 15 is a pneumatic schematic diagram of an alternate system showing a different valving scheme compared to the system in FIG. 1.

FIG. 16 is a pneumatic schematic diagram of an alternate system using a 4-way valve.

FIG. 17 is a pneumatic schematic diagram of an alternate system using a second sensor at the inlet through which all collected gas will flow and be measured.

FIG. 18 is a pneumatic schematic diagram of an alternate system using two pumps, reducing the number of valves required on the inlet side of the sensor, and reducing the pump acoustical sound emittance.

FIG. 19 is a pneumatic schematic diagram of an alternate system to that of FIG. 18, in which an inlet sensor is included.

FIG. 20 is a pneumatic schematic diagram of an alternate system with check valves a second sensor at the inlet, and a differential pressure transducer measuring the input and output pressures.

FIG. 21 is a pneumatic schematic diagram of an alternate system with a dual lumen collection cannula, and optionally an oxygen therapy option.

FIG. 22 is a pneumatic schematic diagram showing filtration and purge features, shown in the state of purging path 2, and optionally an oxygen therapy option and optionally a disinfection light.

FIG. 23 is a line drawing of a collection cannula used with the system shown in FIG. 1.

FIG. 24 is a detailed hidden line view of the portion A of the tip section of the collection cannula in FIG. 23.

FIG. 25 is a hidden line drawing view of the tip section of the cannula of FIG. 23 in which the atmospheric opening to the flow channel is on the side of the cannula rather than at the tip's end.

FIG. 26 is a cross section of section B-B of FIG. 24, showing a dual lumen construction.

FIG. 27 is a cross section of section C-C of FIG. 24, showing a larger diameter compared to the diameter at section B-B.

FIG. 28 is an isometric view of the face and head section of the cannula of FIG. 23, with a face pad and cushion to position and hold the cannula tip in place.

FIG. 29 is a block diagram of the overall system of the invention.

### DETAILED DESCRIPTION

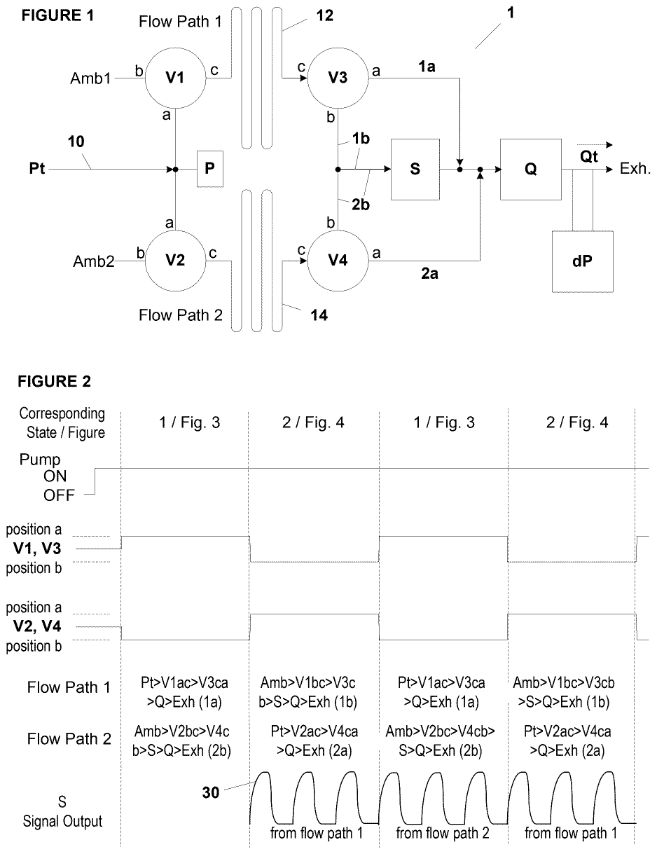

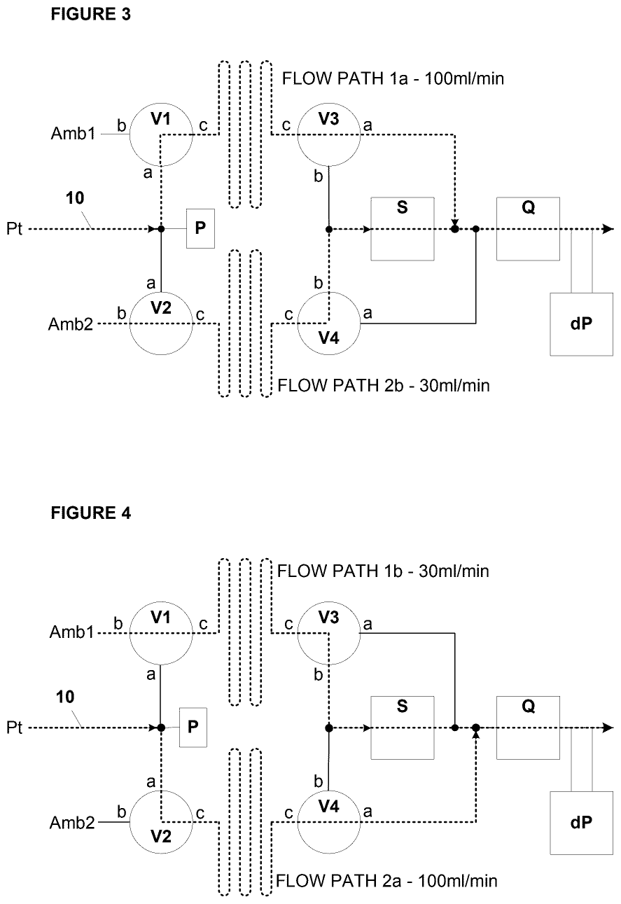

In FIG. 1, the main embodiment of the system is described, using measurement of the CO2 in a breath sample as an example. Gas is draw into the apparatus 1 from the patient Pt through a collection cannula 10, via a pump Q and out through an exhaust Exh. Valves are used to control the flow path of the gas. In the case shown in the figure, the valves are 3-way valves with an always open common port, c, and one other open port, either a or b at any given time depending on the control, and Amb1 and Amb2 are ambient air inlets. There are two sets of two paths, one set flowing while the other set is not flowing, and the two sets alternate. A flowing path is denoted by a dotted line, and a non-flowing path, or a flow path in resting state, is denoted by a sold line, in this figure and throughout the specification. Specifically, in a first state, or state 1, which is described in FIG. 3, the first set of two open paths that are flowing are:
patient collection path 1a: Pt→10→V1ac→12→V3ca→Q→Exh; and
sample measurement path 2b: Amb2→V2bc→14→V4cb→S→Q→Exh.
   where V1ac connotates ports of valve V1 a and c are open and port b is closed. This valve port naming convention follows throughout. The other set's paths are closed during this first state. In the patient collection path 1a, an amply sized volume of breath sample is being collected in the collection conduit 12, while in the sample measurement path, the sample previously drawn into the collection conduit 14 is drawn through the sensor S for measurement. Importantly, the resistance of the measurement path is considerably more than the resistance of the collection path. That makes the flow rate in measurement path relatively slow, for example 30 ml per minute, and the flow rate in the collection path relatively fast, for example 100 ml per minute. 100 ml per minute collection allows for collection of a large volume, and measurement at 30 ml per minute gives the sensor, with its particular response time, enough time to measure the sample that was previously collected. When an adequate amount of sample has entered collection conduit 12 and the sample from collection conduit 14 has been measured, it is time to switch states, which typically occurs without delay or without discontinuation of movement of any of the gas. In FIG. 4 this opposite state, the second state or state 2, is shown and is as follows, with the state 1 paths now closed:
patient collection path 2a: Pt→10→V2ac→14→V4ca→Q→Exh; and
sample measurement path 1b: Amb1→V1bc→12→V3cb→S→Q→Exh.

A control system and algorithm cause the system to alternate between the first and second state. While state 1 is active, the sample previously collected from state 2 is being measured and a new sample is being collected, and when state 2 is active, the sample previously collected from state 1 is being measured and a new sample is being collected, and so on. A gas sample collected in one path branch is measured in the other path branch typically without delay after it is collected, when the algorithm switches states. The frequency and timing of switching between the two states is usually done based on the breath rate, but can also be pre-programmed into the algorithm. In an optional algorithm, the states switch immediately after the complete measurement of a sample traveling through the sensor—when the measurement is complete, the algorithm switches states and begins to measure the collected sample that was previously collected in the other path. This might be after one sample is completely measured, or when two or more consecutive breath samples are completely measured. The switching algorithm can also be determined based on the measured signal and machine learning to find the optimal frequency and timing for which to switch states. A breathing frequency sensor P, capable of detecting a signal characteristic of each breath, can be used to measure a time-based parameter of the breathing signal, which can be used to help control the algorithm to toggle between the state 1 and state 2, and to measure the breath rate, which can be used in the algorithm that determines the frequency or time at which to switch between the two states. Examples of the sensor P are, but not limited to, pressure sensors, CO2 sensors, temperature sensors and flow sensors. Separately, a flow sensor dP, such as a differential pressure sensor or anemometer can be used as a closed loop control measurement to control the pump flow rate to the desired and required flow rate, so that the flow rate and velocity of the gas through the system is precisely know, such that the location of the breaths in the flow pathways is known, so that the breath samples can be properly routed around or through the sensor as required. The flow pathway tubes throughout the system are of especially now friction, to minimize drag of the gas sample along the walls of the tube, to help reduce resistance and reduce mixing cause by frictional drag. The pump Q can be a diaphragm pump, rotary vane pump, or peristaltic pump. The sensor S can be an optical sensor, a chemical sensor, an electrochemical sensor, a mass spectroscopy sensor, an ultrasonic sensor, or other sensor that is especially suitable to measure the substrate in question. If the system is being used to measure carbon dioxide, an infrared sensor can be used. Inlet valves V1 and V2 and sensor inlet valves V3 and V4 are typically low dead space solenoid valves.

The timing diagram in FIG. 2 describes in more detail the flow paths and switching between states 1 and 2, and the function of the pump Q, the position of valves V1 and V3 and valves V2 and V4, and the sensor S output signal 30. It should be noted that will all such systems, there is some latency between when the patient actually exhales a breath, and when the monitor measured that breath, because it takes some time for the sample to travel from the patient's mouth or nose to the sensor. This latency, while not a significant clinical concern, can be minimized in this system which will be described later.

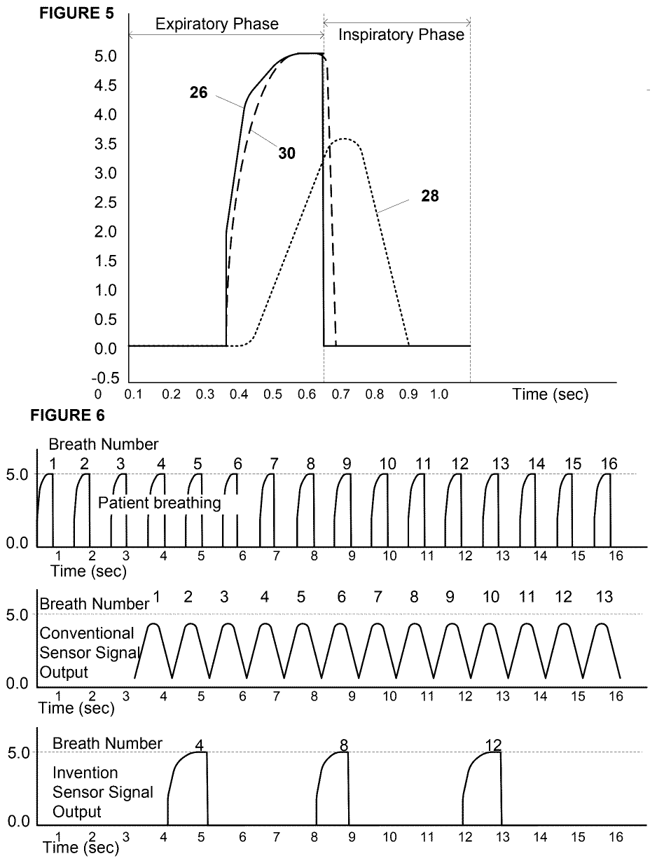

In FIG. 5, a plot is shown to explain the response time of the sensor S in more detail, showing one expiratory period from one breath and the inspiratory period of the next breath. The tracing 26 is the actual true exhaled CO2 level, to which the measured levels should be compared. The tracing 28 is the measured value from the CO2 sensor in a conventional state-of-the-art CO2 measurement system, illustrating how conventional systems are not capable of measuring the peak CO2 during fast breathing frequencies because of the slow overall response time of the sensor relative to the breath signal frequency. The tracing 30 is the measured value from the CO2 sensor in the present invention, showing that the invention results in an accurate CO2 measurement corresponding to the patient's actual CO2 signal. To avoid confusion of the readers of this specification, it is noted that the true level, tracing 26, is theoretical because it actually cannot be measured with currently available equipment on breaths faster than 45 breaths per minute, however the shape of the waveform is trustworthy as it is based on established medical community consensus which is based on various sources of information and decades of research. Arguably, the present invention shown by tracing 30, empirically validates that the medical consensus has been correct.

The graphs in FIG. 6 describe in more detail how the apparatus is able to accurately monitor the patient's CO2, in comparison to a patient breathing at 60 breaths per minute, exhaling an end-tidal CO2 level of 5.0%. The x-axis show time in seconds and the y-axis show percent CO2 concentration. The top graph shows a tracing of the exhaled CO2 as a function of time, measured at the patient's nose over 16 breaths with the patient breathing at 60 breaths per minute. Again, this tracing is theoretical since there is no equipment currently available that responds fast enough to measure CO2 under these conditions. The middle graph shows how the CO2 sensor in a conventional state-of-the-art system attempts to measure the patient's CO2, but is not able to read the peak level because of the responsiveness limitation as previously described. As an aside, it is noted that the conventional system is able to measure breath rate perfectly well, which lends to the confusion in the marketplace on whether or not these conventional systems are compatible with breath rates over 45 breaths per minute. The answer is that they cannot measure CO2 accurately above 45 breaths per minute, but they can measure breath rates up to 90 breaths per minute, however the user is not aware of this nuanced distinction, lending to confusion The bottom graph shows in more detail how the invention shown in FIG. 1 is able to accurately monitor the patient's peak CO2 level. In the first 4 seconds, gas from breaths 1-4 are drawn into branch 1a of path 1, with the fourth breath getting positioned in the collection conduit 12. Then, gas from breath 4 is diverted to branch 1b of path 1, and because branch 1b is more resistive and/or flows gas slower, the gas from breath 4 flows through the sensor S at a much slower flow rate compared to the flow rate at which it was collected, taking 3-4 seconds to draw the complete length of the breath 4 gas sample through the sensor. During the 3-4 seconds that this is occurring, gas from breaths 5-8 is being drawn into branch 2a of path 2, with breath 8 being positioned in the collection conduit 14. After the breath 4 measurement is complete, the system switches flow path states, and breath 8 is drawn into the sensor S through branch 2b of path 2, while breaths 9-12 are being drawn into branch 1a of path 1, setting up breath 12 for subsequent measurement in branch 1b.

In the example provided, this results in a refresh rate, meaning how frequently a CO2 result is reported, of every 4 seconds. The CO2 of some breaths are not measured, which is why the invention is sometimes referred to as a “close-to-continuous”, “quasi-continuous” or “semi-continuous” real time monitor. Skipping the measurement of some breaths is perfectly acceptable clinically, as long as the refresh rate is well within the time required to respond to an adverse clinical event. For example, one breath CO2 result per minute may not be acceptable in some situations because a patient's condition could deteriorate before the clinician would be notified. It should be noted that the above refresh rate is one possibility of the invention, and other refresh rates, number of consecutive breaths being measured and reported, and number of consecutive breaths being missed, are possible and within the scope of the invention. For example, the refresh rate might be 10 seconds, with the CO2 of two consecutive breaths being measured and reported, with 8 consecutive breaths being skipped, and repeating. Again, any combination has been considered in the invention, and the selected combination depends on the clinical application, and other factors like volumetric collection rate, flow path diameter, collection cannula and collection conduit lengths, and pump speed. The refresh rate may also not be a constant—it may modulate up and down based on the patient's breathing pattern or other factors, automatic or manually selected. It is also considered that the system may, under certain circumstances, not toggle between the two states at all and instead may channel gas from all breath samples directly through the sensor S if the patient begins breathing below 45 breaths per minute, for example when the patient matures or becomes healthier.

As mentioned before, it is noted that a latency exists between the time the breath was exhaled by the patient, and the time the measurement is made and reported to the user. For example, breath 4 in FIG. 6 is reported about six seconds after it was expired by the patient. This is inherent and typical in any side stream breath monitoring system because it takes time for the sample to travel through the collection conduit from the patient's mouth or nose to the instrument. In this invention, the initial travel latency of one to eight seconds plus the measurement travel time latency of one to eight seconds, means the CO2 value will be reported to the user two to 16 seconds after the breath was exhaled. The invention is flexible enough so that the overall latency can be well within the clinically acceptable range, and when necessary, the system's control parameters can be set to reduce this latency to the clinical need. Regarding the number of breaths that get skipped and do not get measured, in the example in FIG. 6 three consecutive breaths get skipped between each measured breath, which is a clinically acceptable small number of breaths to ignore. However, it is appreciated in this invention that fewer skipped breaths might be preferred, and as such, alternatives to the main embodiment are described later that minimize breath skipping.

It is also noted that breath rate in the present invention can be accurately measured and reported in a variety of ways as shown in various figures, hence that secondary functionality traditionally expected in a capnometer is not lost as a consequence of measuring peak CO2 levels accurately. In a first technique, the duration of the measured CO signal can be measured, then correlated to a breath rate, since the amount that the signal was slowed down is precisely known. For example, in FIGS. 1 and 6, if the length of the measured CO2 signal is 0.833 seconds long, then the breath rate was 60 breaths per minute ([60/(2.0*2*0.833/3.333)]=60 bpm, where 60 is seconds per minute, 2.0 is exhalation time/CO2 time, 2 is breath period/exhalation time, 0.833 is seconds of CO2 measurement, and 3.333 is collection speed/measurement speed ). Second, there can be a separate breath period sensor somewhere in the flow path, for example a simple pressure sensor P as shown in FIG. 1 which can measure the pressure excursion corresponding to the pressure pulse of each breath, and with that information continuously measure the breath rate. Third, there can be another CO2 sensor in the flow path that measures CO2 in the conventional manner to report the breath rate, as shown in the middle graph in FIG. 6.

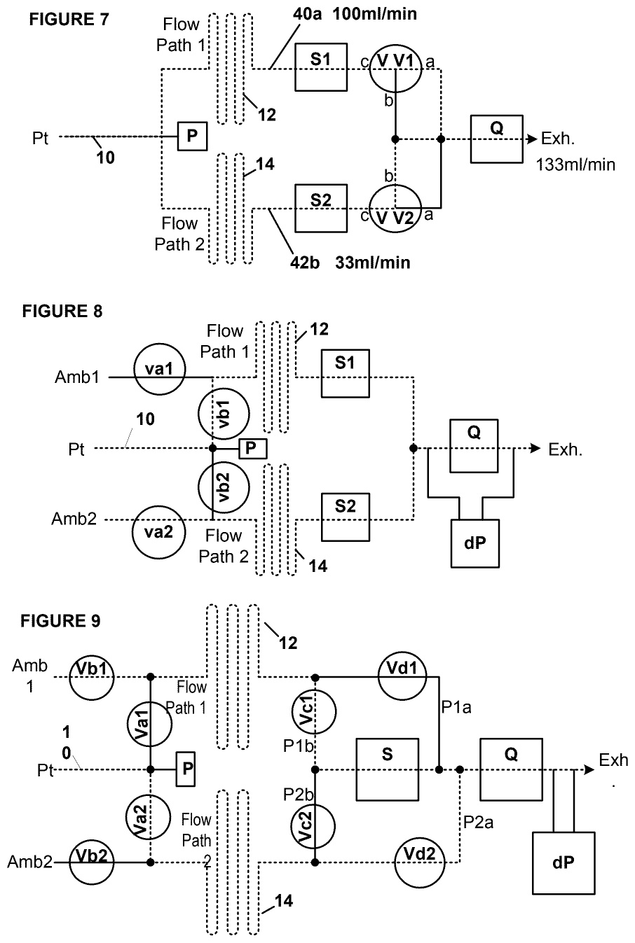

The gas sample from the patient, if flowing through valves, may get mixed in the dead space volume inside the valve, and potentially become diluted, adding to a source of error in the CO2 measurement. As such, this invention includes some novel ways to mitigate such potential effects. First, as shown in FIG. 7, a valve-less system is described which is devoid of valves on the patient side of the sensor S, thus eliminating any opportunity for the gas to get diluted in valves prior to traveling through a sensor as in sensor S of FIG. 1. This embodiment also includes two sensors, S1 and S2, instead of one sensor. Valves VV1 and VV2 control the flow rates through the sensors S1 and S2 by altering the flow paths between valve ports a and b, this toggling between a higher and lower flow rate through each sensor, the lower flow rate being that which to measure the gas sample, and the higher flow rate being that which to collect the gas sample. In a first state shown in FIG. 7, gas from the patient is simultaneously flowing through path 40a and 42b, and in a second state gas from the patient is simultaneously flowing through path 40b and 42a. Paths 40a and 42a, the lessor resistive and faster flow rate paths, are the gas sample collection paths, and paths 40b and 42b, the more resistive and slower flow rate paths, are the gas sample measurement paths. An ample volume of the breath sample gets collected in the collection conduits 12 and 14 at a relatively high speed, for example 100 ml per minute, and on an alternating basis those samples get measured in sensor S1 and S2 at a relatively slow speed, for example 33 ml per minute. These flow rates should be construed as exemplary. In the descriptions, the term speed may be used interchangeably with volumetric flow rate.

Another way to avoid valve dead space volume is to use pincher valves, as shown in FIGS. 8 through 11, instead of valves such as solenoid valves, in which the gas flows through the inside volume of the valve. In the case of FIGS. 8 and 9, the pincher valves are single path valves that have the path either open or closed. The gas does not flow through the internal volume of the valve-instead, a silastic tube is externally pinched by the valve to open or close the tube. The 3-way valve V1 used in the apparatus of FIG. 1 for example is replaced with two pincher valves, va1 and vb1. The tubing used in the pincher valves must have fast recovery time, negligible hysteresis and creep, and resistant to stress fatigue. When in the off or standby state, the pincher valve should be in the open position so that the tube is not pinched closed for unnecessarily long durations of time. In a first state, vb1 is open, va1 is closed, vb2 is closed, and va2 is open, thus the apparatus is collecting breath samples in path 1 and measuring breath samples previously collected in path 2. In the second state, vb2 is open, va2 is closed, vb1 is closed, and va1 is open, thus the apparatus is collecting breath samples in path 2 and measuring breath samples previously collected in path 1. Valves Vc1 and Vc2, and Vd1 and Vd2, replace valves V3 and V4 in the embodiment shown in FIG. 1. The apparatus can be configured such that the tubing can be installed externally to the apparatus, to facilitate periodic replacement of the tubing by the user. This alternative valving approach also guards the inner workings of the monitor from being contaminated with patient gas that might have infectious microbes. FIG. 8 also shows a system with two sensors, S1 and S2 which reduces the number of valves needed, and can reduce the number of skipped breaths as previously described, although an alternative configuration shown in FIG. 9, which is shown in the state of measuring in path 1 and collecting in path 2, can have one sensor and additional pincher valves as necessary.

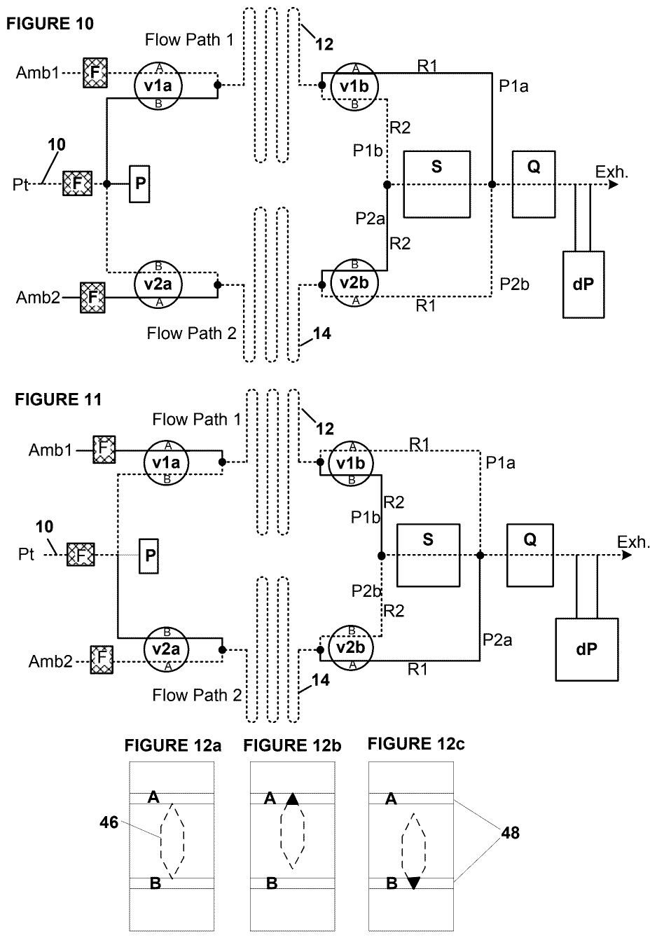

An alternative pincher valve configuration is shown in FIGS. 10, 11, and 12a-c, using two-way pincher valves. These valves have two flow channels, A and B, with tracks 48 where the tubing gets inserted, and a pincher or anvil 46, and typically one channel is pinched and the other channel is open. In a first state shown in FIG. 10, valve v1a channel A is open, valve v1b channel B is open, and valve v2a channel B is open and valve v2b channel A is open. Gas is therefore being collected in path 2 and the gas in path 1 previously collected is being measured. In the second state shown in FIG. 11, the valves states are flipped to open the other channels, and gas gets collected in path 1 and the gas previously collected in path 2 gets measured. The paths Amb1-v1aA-v1bB-S-Q-Exh and Amb2-v2aA-v2bB-S-Q-Exh have a resistance R2. Paths Pt-v1aB-v1bA-Q-Exh and Pt-v2aB-v2bA-Q-Exh have a resistance R1, with R2 being more than R2, so that the measurement speed through S is slower than the collection speed that bypasses S as previously described. Alternatively to the typical pincher valve, these pincher valves can uniquely have three states as described in FIGS. 12a, 12b and 12c, a neutral state (FIG. 12a) in which flow can pass through both path A and B simultaneously, and a channel A state with channel A open and channel B closed (FIG. 12b), and a channel B state with channel B open and channel A closed (FIG. 12c). Or uniquely, the pincher valve can alternatively have two anvils (not shown) such that one anvil can pinch channel A while the other anvil is pinching channel B, so that the valve has a fourth state of both channels closed, in addition to both channels open, channel A/B opened/closed, channel A/B closed/opened. The benefit of this novel pincher valve design is that when off or in standby, the valve is in the neutral state to avoid any unnecessary long-term pinching of one of the tubes, and, if ever needed, both channels can be open for flow.

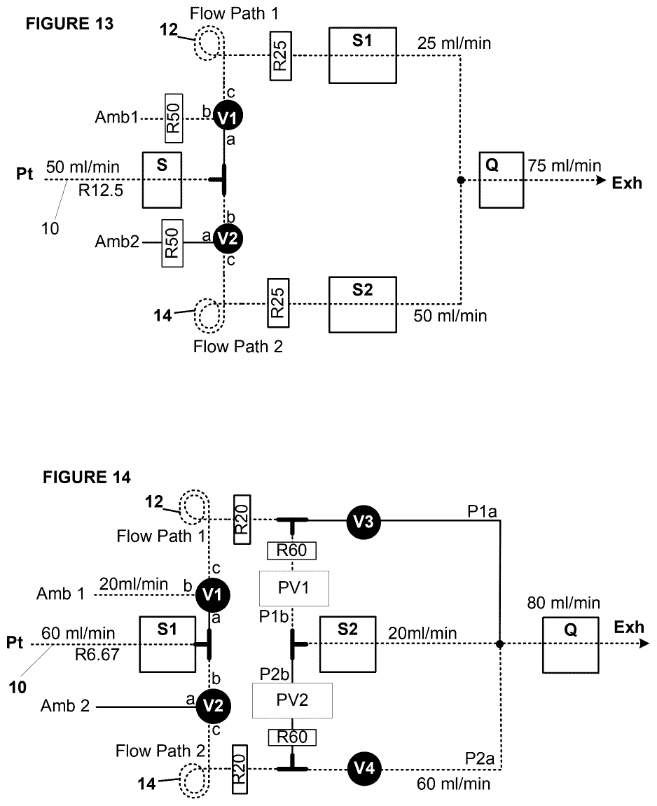

In all figures, where present, analyte or substrate sensors are denoted with an S prefix, pumps with a Q prefix, and valves with a V prefix unless otherwise noted, flow path resistances are denoted with an R prefix, flow paths are denoted with a P prefix, followed by a 1 or 2 corresponding to the pathway, and an a or b suffix denoting which sub flow path, collection or measurement, and flow sensors are denoted dP. As shown in FIG. 13-22, there can be quite a number of different arrangements and quantities of elements of the system including the pump, for example multiple pumps, or multiple analyte or substrate concentration sensors, and different valve arrangements, flow paths, patient inlets, non-patient gas inlets, exhaust ports, filters, and flow rates, and these arrangements are still well within the scope of the invention. In the alternative embodiments that have two or more sensors S1 and S2, one sensor can be used to cross check the other, and vis-a-versa, in order to provide extra reliability. The cross-check routine can be drawing the same sample through both sensors to compare the measurement result, and/or drawing ambient gas through both sensors and comparing the measurement result. If there is a difference in measurement result, a diagnostic function residing in the monitoring system, will cause the sensors to be calibrated. Two sensors S1 and S2 positioned in parallel paths has the additional advantages of dedicated flow pathways for each sensor which can reduce the number of breaths that miss measurement as shown in FIG. 2. In addition, two sensors eliminate one valve upstream of the single sensor configuration, which reduces the opportunity of gas mixing resulting in an even more pure sample. In the alternative embodiments that have two pumps, the advantages are that the maximum flow rate produced by each pump can be less than that of a single pump system, thereby reducing the sound emittance from the device from the pump, and two pumps reduces the number of valves needed. A two parallel pump and two parallel sensor arrangement (not shown, but contemplated), can benefit from the advantages of both the dual sensor and dual pump.

In FIG. 13 a configuration is shown in which there are sensors S1 and S2 in both flow paths. The system is shown in the state of measuring a sample collected in flow path 1, Amb1-V1bc-S1-Q, at a flow rate of 25 ml/min, while simultaneously collecting samples in flow path 2, Pt-S-V2bc-S2-Q. Exemplary collection and measurement flow rates, and flow path resistances are shown, but should be considered exemplary only, and other flow rates are sometimes used depending on the sensor's limit of detection, and signal response time. The limit of detection refers to the amount of substrate in the sample, for example the number of CO2 molecules in the breath gas sample, that can be accurately and reliably detected and measured by the substrate sensor. Ideally, the number of molecules in the sample will be at least 50% greater than the limit of detection to assure that the sensor will perform a reliable measurement of the substrate. Therefore, the collection flow rate is set to be a flow rate that acquires a volume of breath gas that will contain the number of substrate molecules 1.5 times the limit of detection, based on the lowest concentration of the substrate suspected in the gas sample. For example, if the invention is used to measure CO2 in exhaled breath, and the lowest CO2 reading is expected to be 30000 parts per million, and the limit of detection of the sensor is 1.0% CO2 concentration in a 1 ml sample of gas, the collection flow rate can be determined in order to collect 1.5 times the necessary molecules of CO2. An inlet substrate sensor S is included in this design, to measure frequency related parameters of the breath signal and substrate within the breath gas, and this information is used as inputs to the algorithms that control the valves and flow paths, so that the desired breath gas samples are traveling through the branches of the system in the desired and optimal manner. In FIG. 14 a combination of valve types strategically positioned to optimize sample purity, are shown, with 3-way solenoid valves V1 and V2 positioned upstream of the sensor S2, and pincher valves PV1 and PV2 positioned between sensor S2 and the solenoid valves, and 1-way solenoid valves V3 and V4 positioned in parallel with the sensor S2. Again, an inlet substrate sensor S1 is used in this embodiment for the purposes described earlier. Exemplary flow path resistors and flow rates are shown.

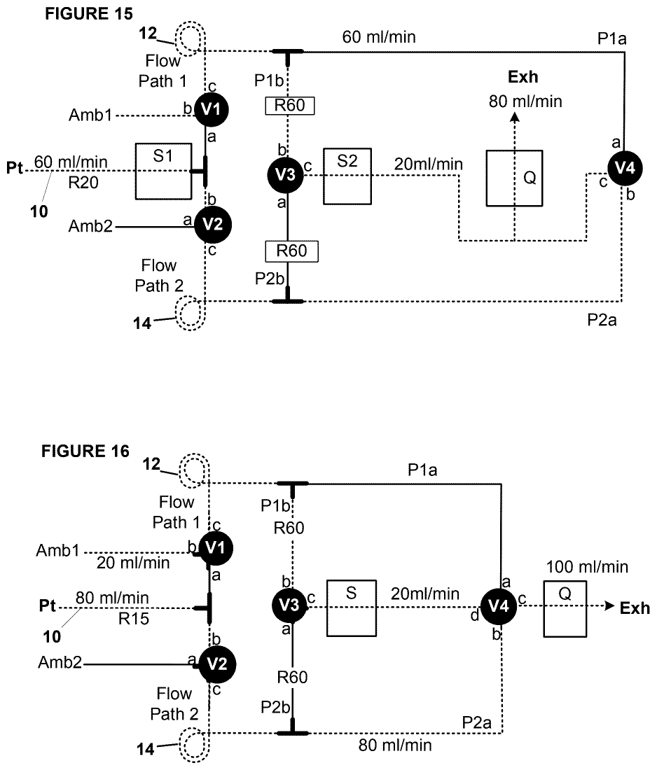

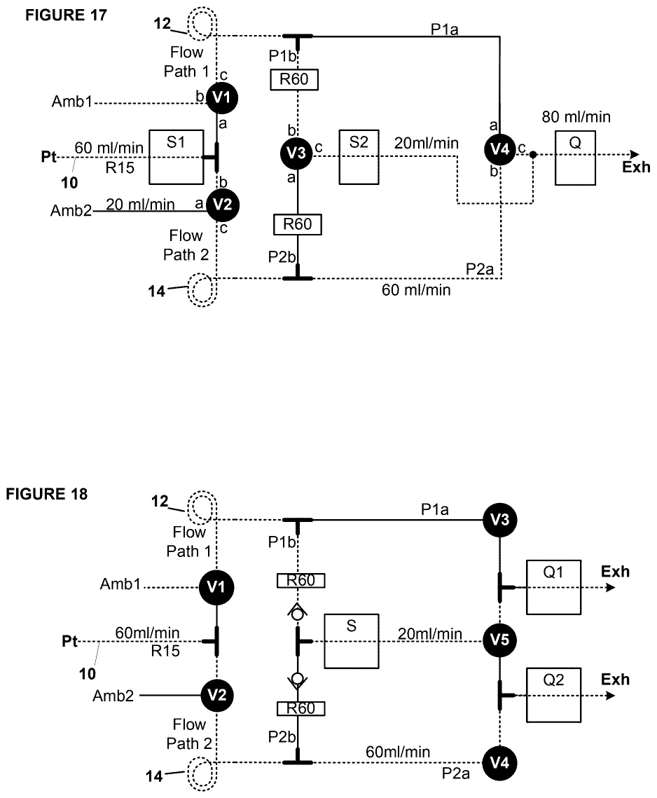

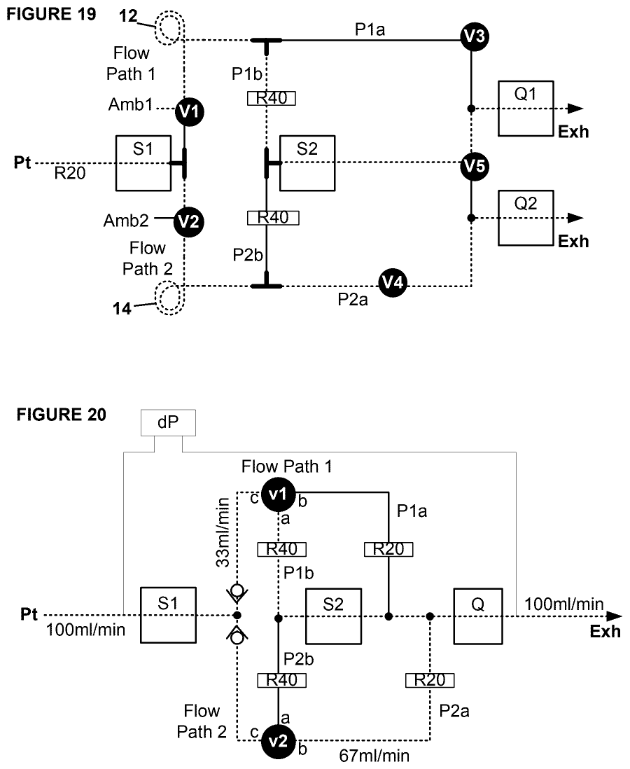

In FIG. 15 an alternate configuration is shown with an inlet substrate sensor S1, and two valves on either flow path upstream of the second substrate sensor S2. In FIG. 16 an alternative pneumatic system is shown with a 4-way valve assisting in the control of the measurement and collection flow paths, shown with flow path 1 in sample measurement mode and flow path 1 in sample collection mode. The pneumatic system in FIG. 17 is configured for a total flow rate of 80 ml/min, with a collection flow rate from the patient of 60 ml/min, and a measurement flow rate of 20 ml/min. FIG. 18 describes a system with two pumps Q1 and Q2, for the advantages stated earlier, with only one valve, V1 and V2, on each pathway upstream of the sensor S, and one-way check valves on paths P1b and P2b upstream of the sensor to prevent backflow from those conduits into the collection flow paths P1a and P2a. FIG. 19 shows a pneumatic configuration with two substrate sensors S1 and S2 and two pumps Q1 and Q2, to use the advantages described earlier. FIG. 20 describes a pneumatic configuration with no ambient inlets in the sample measurement pathway, as is included in most of the other configurations shown. This configuration simply alternates between flow paths 1 and 2. The measurement pathway, for example P1b, because it is far more resistive than the collection pathway P2a, while measuring the gas in path P1b, very little gas is drawn from the patient gas inlet, and most of the patient inlet gas travels into flow path 2.

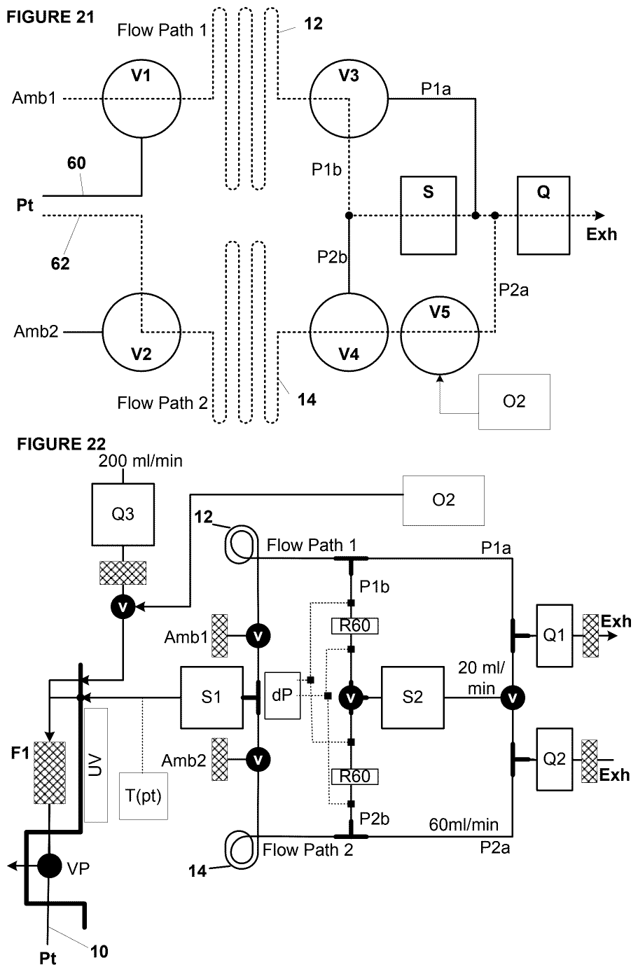

In FIG. 21 an alternative to the main embodiment is shown with two parallel collection cannula channels, 60 and 62, leading into two collection paths and two collection conduits 12 and 14. The two parallel collection channels are typically two lumens in a single tube. The purpose of this embodiment is to have even more options with how to collect and measure the breath. Also shown in FIG. 21 is an alternative embodiment in which a fifth valve is included, V5, for delivery of oxygen to the patient from an oxygen source O2, through one of the collection cannula channels. The oxygen delivery channel can be a dedicated channel, or can toggle between the two channels. The oxygen communicates with the collection cannula or collection cannulas through the appropriate positioning of the valves. In this embodiment, the functions of CO2 monitoring, oxygen therapy, and purging the CO2 monitoring lines (with oxygen) is all accomplished with one device and the appropriate valve control algorithms. One path can be used for CO2 monitoring and another path for oxygen delivery, using a dual lumen collection cannula, or a single lumen collection cannula that alternates.

Because in some cases this invention is used to collect and measure a breath sample or another fluid-based signal from a person, substances from the patient can get into the flow path (the collection cannula, collection conduit, valves, sensors, measurement line and pump), which would degrade performance and contaminate the equipment. The invention takes that into account and in FIG. 22, filtration and flow path purge features are shown. First, filters are provided on inputs and outputs into the equipment. Secondly, periodically, a reverse flow of gas can be sent through the sensors, tubing and valves, etc., toward the patient. This flow will remove any moisture or other debris that has been drawn into the system. Ideally, the purge flow is vented to atmosphere without reaching the patient, to avoid the perception that gas or contaminants are being delivered to the patient. The vent for the purge flow can be another valve, including a pincher valve or solenoid valve, that opens a path to atmosphere while closing off the path to the patient. In a first purge configuration, flow from one of the pumps Q1 or Q2 is reversed to send filtered gas through S2 and S1, and the inlet filter F1, and out the purge vent valve, which will clean out any debris. In a second purge configuration, a third pump Q3 is included which periodically sends flow toward the patient, and out the purge vent valve VP. This configuration may require a more frequent purge duty cycle that the first configuration to avoid debris from getting to the sensor S1. In a third configuration, there is an oxygen source connectable to the system that uses oxygen gas to flush the filter F1, and optionally to deliver oxygen to the patient as previously described. These configurations can be implemented and used separately, or in combination. The purge routine is typically automatic and is activated based on a pre-set automatic routine. One flow path inside the equipment can be getting purged for a few seconds while the other path is undergoing the signal measurement sequence previously described. The next purge routine, for example two minutes later, can purge the other flow path, while the opposite path is being used to measure the sample, and so on. In an alternative embodiment, the pumps Q1 and Q2 can be used to purge the sensors S2 and S1, while the pump Q3 is used to purge the filter F1. Optionally however, there can be a way for the user to manually activate the purge routine, which he or she can do based on some objective criteria, such as observing fluid buildup in the line, or a change in the signal of the measurement that is indicative of debris in the line. Or optionally, the transducers can sense a buildup of resistance in the line, indicative of debris, and either automatically activate the purge routine, or communicate to the user to manually activate the purge routine. It is also given consideration in this invention, that the user will be messaged to remove the collection time from the patient and activate the purge routine, which will take just a few seconds, and then the user can re-insert the collection cannula into the patient. There may also be an ultraviolet light UV to keep the filter F1 clean of any bacteria from entering the apparatus, or being sent back to the patient during a purge. Patient breathing parameters can be measured by transducer T(pt). Flow rate through paths P1b and P2b can be measured with a flow sensor dP. And lastly, it is also given consideration that the purge flow can travel all the way to the patient exit the patient end in a retrograde direction as will be described later. All of the above purge routines can be used individually or in combination.

The filter F1 in FIG. 22 is a low volume humidity and biological filter. The filter can be self-drying by wicking moisture to the outside of the filter. The filter can also be serviceable, allowing the user to access it to clean or replace it. It is calculated that the collection cannula, when including the filter will be able to be used for 24-48 hours before replacement is required, and when including the purge flow feature, will be able to be used for 72 hours prior to requiring replacement. For additional details about FIGS. 13-22, for brevity purposes can be simply cross referenced to the applicable descriptions accompanying FIG. 1-12.

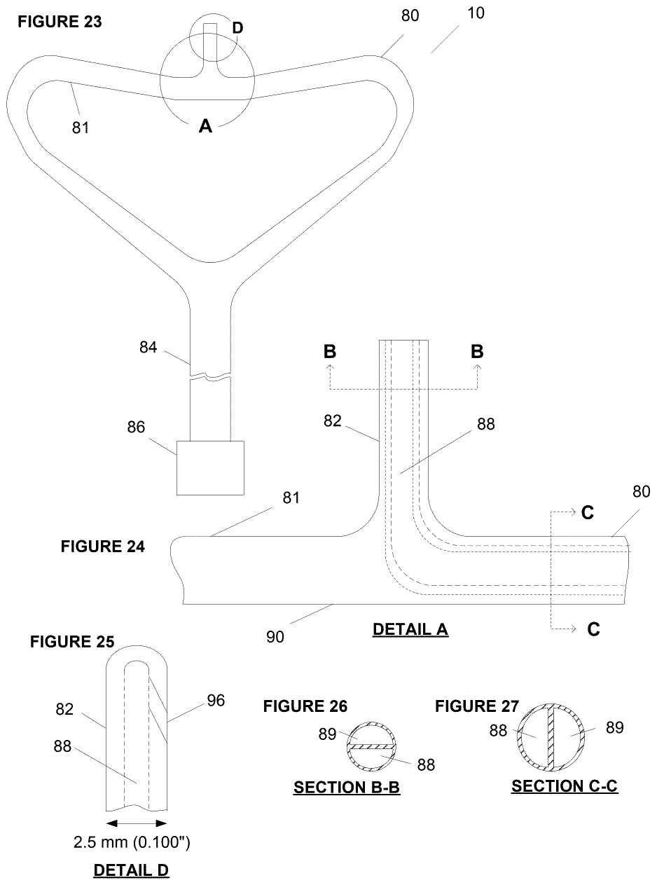

As shown in FIGS. 23 through 28, when the invention is used for measurement of a breath sample, the collection cannula is typically a nasal cannula type device. It can be either a single lumen line for collection of the gas sample, or a dual lumen for collection of the gas sample in both lumens when the apparatus is of the embodiment shown in FIG. 21, or a dual lumen for collection of the gas sample in one lumen and measurement of the breathing pressure in the other lumen, or a dual lumen for collection and measurement of the sample in one lumen and delivery of a therapeutic gas, such as oxygen in the other lumen. The patient end of the cannula includes a gas flow side 80 and a non gas flow side 81, the non gas flow side included principally to festoon the cannula to the patient's head and face. A main cannula section 84 extends the patient end to the machine end 86 which includes the requisite connections and or filters to connect to the apparatus of FIG. 1. A main sample collection lumen 88 is shown in FIG. 24, and a tip section 82 of the patient end, which is inserted typically into the nares, however can be inserted or positioned elsewhere in the breathing pathway. The tip section 82 extends from a manifold base 90. The tip section 82 may terminate with a soft, atraumatic closed tip, and may include a side channel 96 for communication of the gas collection channel 88 to the patient's breathing gas. A novel feature shown in FIG. 25 is included as part of the invention in which the patient end tip of the collection cannula has a retrograde entrance port. Typically, the channel opening is angled away from the patient. This retrograde port helps prevent mucus from entering the flow lumen, as would be the case if the flow lumen terminated in an opening at the very end of the tip. Yet, as breath gas is being exhaled, the vacuum in the retrograde tip easily still draws in the exhaled gas. Additionally, the retrograde tip can be used with the collection cannula purge feature described in FIG. 22, in which case a purge flow is periodically applied to the collection cannula in the direction of the patient, and it travels the entire length of the collection cannula and exits at the retrograde port, shooting away from and not in the patient. The collection cannula can also be removed from the patient by the operator, when the purge procedure is performed to assure nothing is delivered to the patient, or the purge can be timed with exhalation so the patient does not inhale the purge flow. At any rate, the retrograde tip has a number of advantages.

Optionally, the cannula may possess a secondary lumen 89 shown in FIGS. 26 and 27, for the purpose of either a second gas collection channel, or for breathing measurements, or for therapeutic gas delivery, or to alternate with the channel 88 while channel 88 is being purged. As shown in FIG. 24, the tip section may have a slightly smaller diameter than the diameter of the rest of the cannula, so that the tip can fit into the nostril of the neonate, yet the non-tip section is a bit larger to reduce the resistance of the overall cannula, to reduce the amount of drag on the inside cannula flow channel wall. The larger the diameter, the less the ratio between cross sectional area and wall perimeter thus reducing the drag and increasing the linear flow profile. Ideally, the tip section is about 0.020″ to 0.040″ flow channel while the non-tip section is about 0.030″ to 0.050″ flow channel, and most preferably about 0.030″ and 0.040″ +/−0.004″ respectively. In an additional alternative, the tip section has a single lumen, which transitions to a dual lumen at a short distance from the very tip, the dual lumen extending all the way to the equipment, facilitating a double path collection and measurement system such as that shown in FIG. 21. The single path at the very tip allows a smaller outer diameter and insertion into the neonates'nostril, and for the section of tubing outside the nostril, the outer diameter can be larger to no disadvantage.

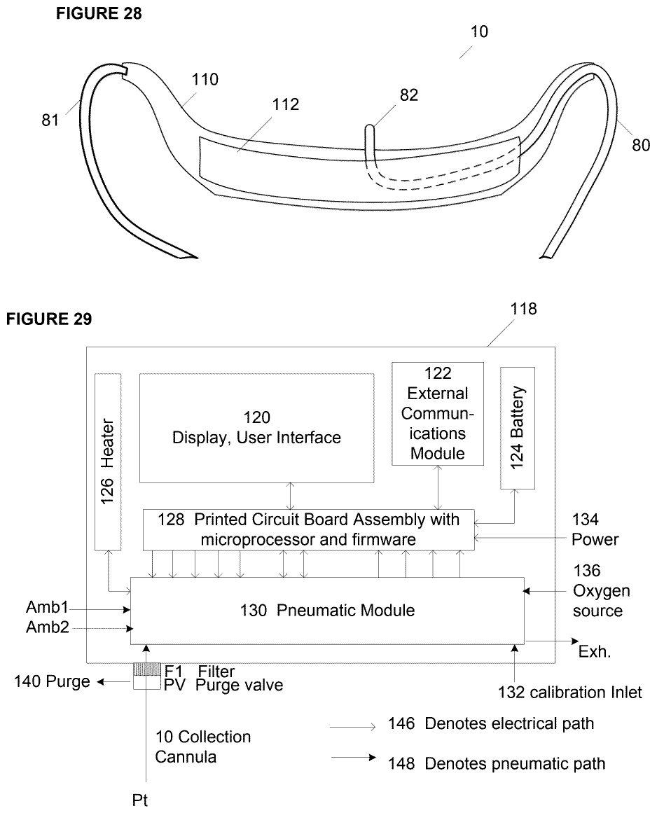

Referring to FIG. 28 a collection cannula fastening device 110 is described in order to attach the collection cannula to the patient. The fastening device is a soft, transparent cushion that is placed under the patient's nose and extending bi-laterally to the cheeks and toward the ears or back of the neck. The collection cannula tubing is embedded under a cover 112 in part of the cushion, to secure the cannula to the cushion, with the tip section of the collection cannula tubing extending above the cushion such that it can be inserted into the nose. Because the collection cannula will have to be on the patient for sometimes up to seven days, it is necessary to have a way of securely attaching it to the face, but with the needed comfort and softness. The tip may include a clip to clip gently to the nose (not shown).

Returning to FIG. 22, the invention also gives consideration to a sensor calibration routine, in which ambient air is drawn into the device by pumps Q1 and Q2 which are switched to reverse flow, and into the sensor S or sensors S1 and S2, and the baseline level of the sensor's signal response is reset. This can be done at power on, or on periodic intervals when in use, such as once an hour. Additionally, in addition to air being drawn in for a baseline or zero-point calibration, a pre-mixed mixture can be drawn in for a non-zero-point calibration data point. The calibration feature applies equally to the other figures such as FIG. 1, with the requisite changes.

In FIG. 29 the overall system is described, including an enclosure 118 housing the device, a user interface 120, a printed circuit board assembly with a microprocessor or CPU and firmware 128, running on a real time operating system, a pneumatic module 130 such as described in FIGS. 1 through 22, ambient inlets amb1 and amb2 for the sample measurement pathway, inlet for patient Pt gas, heater 126, purge flow 140, purge valve PV, filter F1, collection cannula 10, calibration inlet 132, exhaled gas port exh., oxygen source inlet 136, power input 134, and wherein 146 denotes an electrical path and 148 denotes a pneumatic path.

There are other aspects of the invention to note. When used for breath composition measurements, this invention can be used with spontaneously breathing patients, but also with mechanically ventilated patients, for example conventional mechanical ventilation, mask ventilation, and high frequency ventilation. Gas flow can be returned to the patient through the collection cannula to replace the gas taken away from the patient from the measurement. This gas can be the same gas that was removed, or it can be a replacement gas, such as oxygen. The system can enter modes in which there is simply one path collecting and measuring the sample, in the event the frequency of the signal in question slows down, as previously mentioned. The system can be synchronized with another biological function, to make it more comfortable for the patient or more effective. The invention in addition to measuring the amplitude of a signal with a fast frequency, can also be used to measure weak frequencies, or just the frequency itself. A light-based disinfection element may be included where the collection cannula connects to the equipment to help keep the equipment from becoming contaminated. A heater may be included to keep the sample at a relatively constant temperature before it is measured, to prevent condensate from building up in the collection cannula or flow pathways. The heater may be a wire in the collection cannula, and heating element inside the enclosure of the apparatus. The system can acquire the sample with a side stream parallel sampling approach or a main stream in series sampling approach. The pump or collection mechanism speed can be variable, the speed being varied based on the prevailing conditions. The resistance of the pathways can be modifiable to meet the exact flow rate needs, the system can draw in a reference sample either for deliberate dilution of the patient sample for volume amplification or for cross referencing. The pump speed, pathway states, purge routines, and oxygen delivery if included, can be synchronized with the patient's breathing cycle. When used for breath gas monitoring the system can also be used to measure O2 and NO and other respiratory gases, natural or therapeutic.

The apparatus may measure a dynamic signal in a specimen, the signal having at least one periodicity, at least one frequency, and at least one composition peak amplitude, the apparatus comprising: a specimen collection element (e.g., a pump); a specimen inlet; a first and second non-specimen inlet; a signal periodicity sensor; a signal composition sensor; a first and a second collection chamber to gather the specimen; a collection cannula communicating the specimen source to the specimen inlet; a first path comprising: a first branch with a first flow resistance, and with an opened and closed state, and connecting the specimen inlet through the first collection chamber to the collection element, a second branch with a second flow resistance less than the first flow resistance, and with an opened and closed state, and connecting the first non-specimen inlet through the first collection chamber, and through the signal composition sensor to the collection element; a second path comprising: a first branch with a first flow resistance, and with an opened and closed state, and connecting the specimen inlet through the second collection chamber to the collection element, with a first flow resistance, a second branch with a second flow resistance less than the first flow resistance, and with an opened and closed state, and connecting the second non-specimen inlet through the second collection chamber, and through the signal composition sensor to the collection element; an algorithm to control the first and second path's first and second branches, wherein the algorithm comprises: a first state wherein the first path's first branch and second path's second branch are in the open states, and the other branches are in the closed states, and a second state wherein the second path's first branch and first path's second branch are in the open states, and the other branches are in the closed states, and wherein the algorithm alternates between the first and second state.

The apparatus may measure a gas composition in a breath specimen, comprising: a sample collection flow rate generator; a gas composition sensor comprising a response time; a sample collection pathway with a first collection flow rate; a sample composition measurement pathway traveling through the gas composition sensor with a second flow rate, the second flow rate less than the first flow rate; wherein the first flow rate is selected to collect a first volume of gas from at least one breath period, and the second flow rate is selected to play the first volume of gas at a speed equal to or less than the response time of the gas composition sensor. The apparatus may measure a substrate composition in physiological sample, comprising: at least one collection element; at least one substrate composition sensor; at least one collection medium; a first path connecting the at least one collection element, sensor and medium; a second path connecting the at least one collection element, sensor and medium; a first state simultaneously collecting a volume of sample in the first path and measuring the substrate with the sensor in a volume of sample previously collected in the second path; a second state simultaneously collecting a volume of sample in the second path and measuring the substrate with the sensor in a volume of sample previously collected in the first path; an algorithm switching between the first and second state. The apparatus may measure a signal in a specimen, comprising a first collection speed and a second measurement speed, the measurement speed less that the collection speed. The apparatus may measure the gas composition in exhaled breath, comprising two parallel gas flow pathways, a first pathway alternating between collecting the exhaled breath and measuring the exhaled breath, and a second pathway alternating between measuring the exhaled breath and collecting the exhaled breath.

The apparatus may comprise: a gas inlet, a gas sensor, a pump, a pneumatic pathway 1 between the gas inlet and pump and a pneumatic pathway 2 between the gas inlet and pump, pathway 1 and 2 in parallel, at least one of the pathways comprising a valve, and wherein pathway 1 and 2 each comprise two parallel conduits, path-conduits 1-A and 2-A through the gas sensor and path-conduits 1-B and 2-B around the gas sensor, wherein conduits 1-A and 2-A comprise a first flow resistance RA, and conduits 1-B and 2-B comprise a flow resistance RB, wherein RA is greater than RB, a tube with a machine end connectable to the gas inlet and a patient end adapted to be placed in communication with a patient airway, a control system adapted to: draw breathing gas from the tube into pathway 1 and 2, and control the valve to alternate the gas flow path between a first mode and a second mode, the first mode controlling gas to flow through conduits 1-A and 2-B simultaneously, and the second mode controlling gas to flow through conduits 1-B and 2-A simultaneously. The apparatus may comprise: a gas inlet, a breathing sensor, a gas sensor, a pump, a valve, a pneumatic pathway connecting the gas inlet, breathing sensor, gas sensor and pump, a tube with a first machine end connectable to the gas inlet and a second patient end adapted to be placed in communication with a patient airway, a control system adapted to control the pump and/or valve to draw a volume of breathing gas into the gas inlet through the breathing sensor at a first flow rate, and to control the pump and/or valve to draw the volume of breathing gas through the gas sensor at a second flow rate, wherein the second flow rate is less than the first flow rate.

The apparatus may comprise a sampling tube and a measurement apparatus, wherein the sampling tube comprises a patient end in communication with a breathing pathway, and a machine end in communication with the apparatus, the apparatus comprising: a pump in communication with the sampling tube continuously drawing gas from the breathing pathway, a control system with an algorithm controlling the pump speed, a breath composition sensor, and further wherein the algorithm controls drawing gas from the breathing pathway at a first flow rate and measures gas at a second flow rate, wherein the second flow rate is less than the first flow rate, wherein the apparatus is adapted to slow the flow rate down during compositional analysis by the sensor to make the breath appear slower than its original speed. The apparatus may comprise a sampling tube and a measurement apparatus, wherein the sampling tube comprises a patient end in communication with a breathing pathway, and a machine end in communication with the apparatus, the apparatus comprising: a sampling pump in communication with the sampling tube continuously drawing gas from the breathing pathway, a control system and algorithm with a first and second sequence controlling a 1st and 2nd valve, a first and second sampling pathway each with an inlet end and outlet end, wherein the pathways are in parallel with each other and connected by at least one valve at the inlet ends and at least one valve at the outlet ends; a branch between the two pathways connecting the sensor and pump; a breath composition sensor between the inlet end and outlet end valves; and further wherein the algorithm controls the inlet and outlet end valves such that in the first sequence gas is moved by the pump through the first pathway at a first flow rate traveling through the inlet and outlet valves around the sensor while gas is moved through the second pathway at a second flow rate through the inlet and outlet end valves and through the sensor, wherein the second flow rate is less than the first flow rate and in the second sequence.

The specimen may be a breath sample and the signal may be a carbon dioxide concentration signal. The sample collection and measurement may be selected from the group of: a breath sample, a blood sample, a neurological signal, a vascular signal, a cardiac signal, a cellular activity signal, a genetic process signal, or an electrophysiology signal. The measurement sensor may be chosen from the group, a light wavelength sensor, a chemical sensor, a carbon nanotube sensor, an electrochemical sensor, a spectrophotometer sensor, a mass spectrometry sensor, an ultrasonic sensor. The composition sensor may be an infrared sensor and the breath period sensor is selected from the group: infrared sensor, pressure sensor, thermal sensor. The gas composition sensor may be a CO2 sensor. The system may comprise two sensors. The pump flow rate may be X, and the collection flow rate may be Y, and the measurement flow rate may be X-Y. The collection speed may be 100-200 ml per minute and the measurement speed may be 20-50 ml per minute. The first collection flow rate may be high enough to obtain >0.2 ml of exhaled gas from a breath of a breath frequency of 60 breaths per minute, and second measurement flow rate may be low enough to elongate the exhaled gas sample exhalation period to >1 second. The collection conduit and collection cannula tube may comprise a lubricious material to create a low coefficient of friction between the sample and the tube, such as but not limited to, Teflon, polypropylene, plasma treated PVC, plasma treated silicone, heparin treated plastic or thermoplastic, surfactant treated plastic or thermoplastic, polyurethane. The internal flow channel diameters of the collection cannula and collection conduit may be 0.020 to 0.060 inches diameter, and preferably may be 0.030 to 0.040 inches diameter. The flow path control valves may be a pincher anvil that pinches the tube.

At least one flow path control valve may be a pincher valve with two tube channels into which to flow path tubes are inserted, with an anvil, and three states, a first state in which the anvil occupies a first tube channel to pinch a flow path tube, and a second state in which the anvil occupies the second tube channel to pinch a flow path tube, and a third state in which the anvil does not occupy either tube channel. At least one flow path control valve may be a pincher valve with two tube channels into which to flow path tubes are inserted, with two anvils, and four states, a first state in which one anvil occupies a first tube channel to pinch a flow path tube, and a second state in which one anvil occupies the second tube channel to pinch a flow path tube, and a third state in which the both anvils do not occupy either tube channel, and a fourth state in which one anvil occupies one tube channel and the other anvil occupies the other tube channel.

The system may comprise two pumps. The collection cannula may comprise two parallel gas flow channels, a first channel connected to the first path and a second channel connected to the second path. The system may comprise a filter at the collection cannula inlet to the apparatus. The system may comprise a purge pump, a purge line, and a purge opening, and a purge algorithm to deliver gas through the purge line through the purge opening. The system may comprise collection cannula patency features, the features comprising (1) a filter in line with the collection cannula, (2) a pump to generate a purge flow connected to the collection cannula, the purge flow a flow of gas directed away from the device and toward the patient in the collection cannula, (3) a valve communicating with the collection cannula on the patient side of the filter, wherein the valve opens to atmosphere during the purge. The collection cannula patient end tip end may be closed to flow and the tip section includes a side channel communicating the flow channel inside the tube to the outside. The system may comprise a calibration line, calibration valve, and calibration algorithm, the algorithm controlling a pump and calibration valve to draw in ambient air into the measurement sensor, and to reset the baseline signal output of the measurement sensor to equate to the ambient air composition.

The apparatus may be a side stream collection and measurement apparatus, in parallel with the patient's exhaled gas flow. The apparatus may be a main stream collection and measurement apparatus in line with the patient's exhaled gas flow. The apparatus may be adapted to measure a substrate in the patient's exhaled breath when the patient is spontaneously breathing. The apparatus may be adapted to measure a substrate in the patient's breathing gas when the patient is receiving supplemental ventilation for a supplemental respiratory support device. The collection cannula may be adapted to be inserted into the body, for example into the patient's lungs. The system may include an oxygen delivery module, delivering oxygen to the patient continuously through a parallel channel, or cyclically by alternating the oxygen delivery with the sample collection.

The system may measure a first breath sample, and after the measurement is complete, the algorithm switches to the opposite state. The algorithm may switch states based on one or more of the following: the number of breaths measured in a state, the number of breaths collected in a state, a breathing frequency parameter, a characteristic amplitude aspect of the measured signal, a characteristic frequency aspect of the measured signal, the position of the sample in the sample collection cannula based on the breathing sensor data, a pre-set duration, a user-settable duration. The first and second state may comprise a beginning and an end, the first state beginning defined by a first characteristic of a breath, and the first state end defined by a second characteristic of a breath. A system may report the breath rate, the breath rate being determined by the breathing sensor, or by an algorithm related to the sample measurement. The system may comprise four pathways connecting the patient's airway to a pump, pathway 1a and 1b and pathway 2a and 2b, 1a and 2a bypassing a measurement sensor and 2a and 2b traveling through a measurement sensor. The system may measure a substrate in a breath gas sample, and may comprise: (a) a collection cannula, (b) a valve, (c) a collection conduit, (d) a measurement line, (e) a substrate measurement sensor, (f) a pump generating a flow rate of speed X, (g) an algorithm; further wherein (1) the collection cannula collects the sample at a first speed of Y, and (2) the valve introduces non-patient gas at a speed of X-Y, and (3) the measurement line and sensor measures the sample gas mixed with the non-patient gas at a speed of X.

In addition to devices and systems described above, the invention also applies to the method of measuring a physiologic signal in a physiologic specimen, the method consisting of the steps of (1) collecting the specimen at a first speed, and (2) measuring the signal at a second speed, the measurement speed based on the response time of the sensor being used to measure the signal, and less than the collection speed. In the method the specimen may be a breath specimen and the signal may be a carbon dioxide concentration signal. The embodiments described above can be taken individually or in any combination, and be within the contemplated scope of the invention.

1. An apparatus for measuring a substrate concentration in a breath gas specimen selected from multiple breaths, comprising:
a) a breath sample collection gas flow generator;
b) a breath substrate concentration measurement sensor comprising a measurement response time, and a limit of substantially accurate detection;
c) a control system;
d) a first breath gas flow pathway used for breath sample collection, the pathway and control system producing a first breath gas flow rate, wherein the first flow rate is selected to collect a defined volume of breath gas from one breath within the multiple breaths, and in which the defined volume is selected to be of a volume containing a sufficient number of said substrate molecules to be over the substantially accurate detection limit of the substrate concentration measurement sensor;
e) a second breath gas flow pathway used for substrate concentration measurement traveling through the substrate composition sensor, the pathway and control system producing a second breath gas flow rate, the second flow rate less than the first flow rate, wherein the second flow rate is selected to move the volume of breath gas at a speed approximately equal to or less than the response time of the substrate concentration sensor.

2. An apparatus as in claim 1 comprising a gas inlet, a collection cannula with a machine end connectable on a first end to the gas inlet and adaptable to a patient airway on the second end, wherein the pathways comprise pathway 1a and 1b and pathway 2a and 2b, 1a and 2a bypassing the breath substrate sensor and 2a and 2b traveling through the breath substrate sensor.

3. A breath monitoring apparatus for measuring a substrate in exhaled gas, comprising:
(a) a breath gas inlet,
(b) a pump,
(c) a gas substrate sensor positioned between the breath gas inlet and pump,
(d) a pneumatic pathway 1 between the gas inlet and the pump, comprising two parallel conduits, sub-path 1B through the substrate sensor and sub-path 1A around the substrate sensor, sub-path 1B of flow resistance RB and sub-path 1A of flow resistance RA, resistance RB greater than resistance RA;
(e) a pneumatic pathway 2 between the gas inlet and the pump, comprising two parallel conduits, sub-path 1B through the substrate sensor and sub-path 1A around the substrate sensor, sub-path 2B of flow resistance RB and sub-path 2A of flow resistance RA;
(f) at least one flow path flow control valve connected to pathway 1 and/or pathway 2;
(g) a collection cannula with a machine end connectable to the gas inlet and a patient end adapted to be placed in communication with a patient airway,
(h) a control system adapted to:
(i) draw breathing gas, using the pump, from the collection cannula into pathway 1 and 2, and
(ii) control the at least one flow control valve to alternate the gas flow path between a first state and a second state, the first state controlling gas to flow through pathways 1A and 2B simultaneously, and the second state controlling gas to flow through conduits 1B and 2A simultaneously.

4. An apparatus as in claim 3 wherein the breath gas substrate measurement sensor is selected from the group of: light wavelength sensor; chemical sensor; carbon nanotube sensor; electrochemical sensor; spectrophotometer sensor; mass spectrometry sensor; ultrasonic sensor; infrared sensor; CO2 sensor.

5. An apparatus as in claim 3 further comprising a breathing pattern measurement sensor connected to the gas inlet, selected from the group of: infrared sensor; pressure sensor; thermal sensor; the breathing pattern measurement sensor measuring at least one breath pattern parameter selected from the group: breath period; breath rate; expiratory duration; inspiratory duration; a frequency parameter; an amplitude parameter;
and further wherein the first and second state comprise a beginning and an end, the first state beginning defined by a first characteristic of a breath pattern parameter, and the first state end defined by a second characteristic of a breath pattern parameter;
and further wherein the breath rate is reported, the breath rate being determined by the breathing sensor, or by an algorithm related to the substrate measurement.

6. An apparatus as in claim 3 further wherein pathways 1 and 2 comprise, between the gas inlet and the substrate sensor, collection conduits of sufficient length to contain the gas volume from at least one exhaled breath, and further wherein sub-pathways 1B and 2B are connected to ambient gas inlets, the ambient gas inlets positioned between the breath gas inlet and the conduits.

7. An apparatus as in claim 3 comprising a second breath gas concentration sensor, wherein path 1B travels through the first breath gas concentration sensor, and path 2B travels through the second breath gas concentration sensor.

8. An apparatus as in claim 3, comprising a second pump wherein pathway 1 travels through the first pump and pathway 2 travels through the second pump.

9. An apparatus as in claim 3 wherein the pump flow rate is X ml/min, and the collection flow rate is Y ml/min, and the measurement flow rate is (X-Y) ml/min.

10. An apparatus as in claim 3 wherein the collection speed is 100-200 ml per minute and the measurement speed is 20-50 ml per minute.

11. An apparatus as in claim 3 wherein the first collection flow rate is selected to obtain greater than 0.2 ml of exhaled breath gas from a breath of a breath frequency of 60 breaths per minute, and the second measurement flow rate is selected to elongate the breath gas sample exhalation period to greater than 1 second.

12. An apparatus as in claim 3 wherein the collection cannula and pneumatic pathways 1 and 2 comprise internal gas flow channels, the gas flow channels comprising a lubricious material to create a low coefficient of friction between the sample and the flow channel walls, such as but not limited to: Teflon, polypropylene, plasma treated PVC, plasma treated silicone, heparin treated plastic or thermoplastic, surfactant treated plastic or thermoplastic, polyurethane.

13. An apparatus as in claim 3 wherein the internal flow channel diameters of the collection cannula and pneumatic pathways 1 and 2, are 0.020 to 0.060 inches diameter, and preferably 0.030 to 0.040 inches diameter.

14. An apparatus as in claim 3 wherein at least one flow control valve is a pincher valve with two states, and wherein part of the pneumatic pathway 1 and/or 2 comprises a compliant tube, wherein the compliant tube is inserted through the pincher valve, the pincher valve comprising a pinching anvil, the control system comprising a function to move the anvil to pinch the compliant tube thereby shutting off flow through the compliant tube, and to move the anvil to not pinch the compliant tube thereby opening flow through the compliant tube.

15. An apparatus as in claim 3 wherein part of the pneumatic pathway 1 and/or 2 comprises two compliant tubes, and wherein at least one flow path flow control valve comprises (a) two tube channels through which the two compliant tubes are inserted, (b) a pinching anvil, (c) and three states, wherein the control system comprises functions to create a first state in which the anvil pinches a first compliant tube, and un-pinches a second compliant tube, and a second state in which the anvil un-pinches the first compliant tube and pinches the second complaint tube, and a second state in which the anvil occupies the second tube channel to pinch a second complaint tube, and a third state in which the anvil does not pinch either compliant tube.

16. An apparatus as in claim 3 wherein part of the pneumatic pathway 1 and/or 2 comprises two compliant tubes, and wherein at least one flow path flow control valves comprises (a) two channels through which the two compliant tubes are inserted, (b) two anvils, (c) and four states, wherein the control system comprises functions to create a first state in which a first anvil pinches the complaint tube in a first channel and a second anvil is not pinching the compliant tube in the second channel, a second state in which the first anvil is not pinching the tube in the first channel and is pinching the tube in the second channel, a third state in which both anvils are pinching the tubes in both channels, and a fourth state in which both anvils are pinching the tubes in both anvils.

17. An apparatus as in claim 3 wherein the collection cannula comprises two parallel gas flow channels, a first channel connected to the first pathway and a second channel connected to the second pathway.

18. An apparatus as in claim 3 further comprising collection cannula patency features, the features comprising: (1) a filter in line with the collection cannula, (2) a pump to generate a purge flow connected to the collection cannula, the purge flow a flow of gas directed away from the device and toward the patient in the collection cannula, (3) a valve communicating with the collection cannula on the patient side of the filter, wherein the valve opens to atmosphere during the purge.

19. An apparatus as in claim 3 wherein the control system comprises an algorithm to switch states, the algorithm based on one or more of the following: the number of breaths measured in a state, the number of breaths collected in a state, a breathing frequency parameter, a characteristic amplitude aspect of the measured signal, a characteristic frequency aspect of the measured signal, the position of the sample in the sample collection cannula based on the breathing sensor data, a pre-set duration, a user-settable duration.

20. A method of measuring a breath analyte concentration in a breath sample, the method consisting of the steps of (1) collecting the breath sample at a first collection speed, and (2) measuring the analyte at a second measurement speed, the measurement speed selected to be approximately equal to or less than the response time of the sensor being used to measure the analyte, and the collection speed faster than the measurement speed and selected based on a target breath sample collection size.
